(19) United States
(12) Patent Application Publication
(10) Pub. No.: US 2004/0022800 A1
(43) Pub. Date: Feb. 5, 2004

(54) Respiratory syncytial virus vaccine

(76) Inventors: Mark Parrington, Bradford Ontario (CA); Robert J. Sloan, Etobicoke Ontario (CA); Valerie Sales, Toronto Ontario (CA); Judith Atkins, Richmond (CA); Ernst Braendli, Brampton Ontario (CA); Mathilde Luciani, Toronto Ontario (CA); Bernard Cornet, Coopersburg, PA (US); Bruce Carpik, Mississauga (CA)

Correspondence Address: SIM & MCBURNEY, 330 UNIVERSITY AVENUE, 6TH FLOOR, TORONTO, ON, M5G 1R7, CA

(21) Appl. No.: 10/333,839

(22) PCT Filed: Jul. 31, 2001
(86) PCT No.: PCT/CA01/01104

(51) Int. Cl. A61K 39/12

(52) U.S. Cl. 424/186.1

(57) ABSTRACT

An immunogenic composition which may be formulated for protection of a host against disease caused by infection by Respiratory Syncytial Virus (RSV) is provided. The immunogenic preparation comprises at least one protein of RSV or at least one immunogenic fragment of the at least one protein and is not adjuvanted. The at least one RSV protein may be the F, G or M protein from a RSV A or RSV B strain. The compositions may be stabilized for storage. Methods of immunization using the immunogenic preparations are also provided.

### FIELD OF THE INVENTION

The present invention relates to the field of immunology and is particularly concerned with vaccine preparations against Respiratory Syncytial Virus (RSV).

### BACKGROUND OF THE INVENTION

Human Respiratory Syncytial Virus (RSV) is a major cause of respiratory tract infections. Globally, 65 million infections occur every year resulting in 160,000 deaths (ref. 1; a list of references appears at the end of the disclosure and each of the references in the list is incorporated herein by reference thereto.) In the USA alone 100,000 children, may require hospitalization for pneumonia and bronchiolitis caused by RSV in a single year (refs. 2,3). Providing inpatient and ambulatory care for children with RSV infections costs in excess of $340 million annually in the USA (ref 4).

RSV is a major cause of serious lower respiratory illness in elderly and immunocompromised adults (refs. 5 to 9) Outbreaks in nursing or retirement homes are well documented (ref. 10) and a significant proportion of disease involving the lower respiratory tract in outbreaks were associated with mortality. Approximately 35% of hospitalized community acquired pneumonias have been attributed to RSV. Mortality due to RSV may exceed that due to influenza by 60 to 80% (ref. 11) The annual costs attributed to hospitalizations for RSV pneumonia in the elderly in the USA has been conservatively estimated at between $150 to $680 million (ref. 12). An RSV vaccine could therefore play an important role in lessening morbidity and mortality in the elderly and decreasing health care costs.

RSV is an enveloped RNA virus of the family paramyxoviridae and of the genus pneumovirus. The structure and composition of RSV has been elucidated and is described in detail in the textbook “Fields Virology”, Fields, B. N. Raven Press, N.Y. (1996), pp 1313-1351 “Respiratory Syncytial Virus” by Collins, P., McIntosh, K., and Chanock, R. M. (ref. 13).

Cross neutralization studies have shown that RSV isolates can be classified into two major antigenic groups, designated A and B. (ref. 24) The G glycoprotein shows the greatest divergence between groups showing 53% amino acid homology between RSV A and B. (ref.25)

The two major protective antigens of RSV are the envelope fusion (F) and the attachment (G) glycoproteins (ref. 14). The F protein is synthesized as an about 68 kDa precursor molecule (Fo) which is proteolytically cleaved into disulfide-linked F1 (about 48 kDa) and F2 (about 20 kDa) polypeptide fragments (ref. 15). The G protein (about 55 kDa) is heavily O-glycosylated, giving rise to a glycoprotein of apparent molecular weight of about 90 kDa (ref. 16). Two broad subtypes of RSV have been defined A and B (ref. 17). The major antigenic differences between these subtypes are found in the G glycoprotein while the F glycoprotein is more conserved (refs. 4,18).

Antibodies directed against the F protein or against the G protein can neutralize the virus. Antibodies to the F protein block the spread of the virus between cells.

In addition to the antibody response generated by the F and G glycoproteins, human cytotoxic T cells produced by RSV infection have been shown to recognize the RSV F protein, matrix protein (M), nucleoprotein (N), small hydrophobic protein (SH), and the nonstructural protein (lb.) (ref 19).

International patent application WO 94/27636 of Hancock, et al published Dec. 8, 1994 (and incorporated herein by reference thereto) is indicative of approaches to the development of sub-unit vaccines against RSV. This patent application concerns the identification of preferred adjuvants for RSV vaccines; RSV F and RSV G proteins were found to be non-immunogenic in the absence of alum (see Page 19 of WO 94/27636).

Adjuvants have been used for many years to improve the host immune response to antigens of interest in vaccines, especially subunit or component vaccines comprised of recombinant proteins. Adjuvants are immunomodulators that are typically non-covalently (inked to antigens and are formulated to enhance the host immune response. Examples include aluminum hydroxide and aluminum phosphate (collectively commonly referred to as alum). While little or no systemic toxicity is observed with alum, its use is associated with local reactions, such as erythema, subcutaneous nodules, contact hypersensitivity and granulomatous inflammation. Such local reactions may be of particular concern in the context of frequent, for example, annual immunizations, as may be required for the elderly. Thus, it would be desirable to identify vaccine components, such as RSV subunit components, that could elicit a protective immune response in the absence of extrinsic adjuvants, such as alum.

In U.S. Pat. No. 6,020,182, assigned to the assignee hereof and the disclosure of which is incorporated herein by reference, there is described a co-isolated mixture of purified F, G and M proteins of RSV and immunogenic compositions containing the same. The immunogenic compositions were shown to confer protection in an animal model in the presence of extrinsic adjuvants, specifically alum and ISCOMS.

### SUMMARY OF THE INVENTION

The present invention provides non-adjuvanted sub-unit RSV vaccines and methods of making and using the same.

In one aspect, the present invention provides immunogenic compositions (including vaccines), comprising at least one protein of RSV or an immunogenic fragment thereof and a pharmaceutically-acceptable carrier therefor, wherein the immunogenic composition is formulated in the absence of an extrinsic adjuvant. The immunogenic compositions may be formulated as vaccines for in vivo administration for protection of a host, such as a human host, against disease caused by RSV.

The RSV may be an RSV A or RSV B strain and at least one RSV protein may be selected from the group consisting of RSV F protein, RSV G protein, RSV M protein and immunogenic fragments of the RSV F, G or M proteins. In particular embodiments the at least one RSV protein may comprise a mixture of RSV F protein, RSV G protein, RSV M protein. The mixture of F, G and M proteins preferably is provided in the form of a copurified mixture isolated form a strain of RSV. In particular preparations, the mixture of RSV proteins may be present in the relative proportions of:

F from about 40 to about 70 weight %;

G from about 2 to about 20 weight %; and

M from about 20 to about 50 weight %.

In the latter immunogenic composition, when analyzed by reduced SDS-PAGE analysis, said fusion (F) protein comprises F₁ of molecular weight approximately 48 kDa and F₂ of molecular weight approximately 23 kDa, said attachment (G) protein comprises a G protein of molecular weight approximately 95 kDa and a G protein of molecular weight approximately 55 kDa, and said matrix (M) protein comprises an M protein of approximately 31 kDa.

In the latter immunogenic composition, when analyzed by SDS-PAGE under reducing conditions and silver stained, the ratio of F₁ of molecular weight approximately 48 kDa to F₂ of molecular weight approximately 23 kDa is between 1:1 to about 2:1 by scanning densitometry.

In a specific embodiment, the mixture of RSV protein consists essentially of RSV F, G and M proteins, which preferably is free from testing and from monoclonal antibodies.

The protein may be present in the immunogenic preparation in an amount of between about 0.1 micrograms (μg) to about 200 μg per dose. In specific embodiments of the invention, when analyzed under reducing conditions, the F protein comprises heterodimers of apparent molecular weight of about 70 kDa and dimeric and trimeric forms of the RSV F protein, the G protein comprises G protein of molecular weight approximately 95 kDa and G protein of molecular weight approximately 55 kDa and oligomeric G protein and the M protein comprises M protein of molecular weight approximately 28 to 34 kDa.

In a specific embodiment of the invention, the immunogenic composition of the invention may further comprises a stabilizer against storage degradation of the at least one RSV protein. For such purpose, the immunogenic composition may be formulated as a freeze-dried preparation. The storage stabilizer may be a sugar, such as mannitol, sorbitol, sucrose and an L amino acid, such as L-Arginine-HCl, L-Lysine-HCl, L-Methionine, L-Phenylalanine, L-Tryptophan, L-Tyrosine, L-Asparagine, L-Aspartic acid and L-Glycine.

The storage stabilizer employed preferably is sucrose, which may be present in an amount of about 2 to about 10% w/v. Preferably, the sucrose is present in a weight ratio to the mixture of RSV F, G and M proteins of 1:1.

In a further aspect of the present invention, there is provided a method of formulaitng the immunogenic preparations provided herein, comprising:

formulating an immunogenic RSV composition provided herein with a stabilizer against storage degradation;

effecting a freezing step on the resulting formulaiton;

effecting a primary drying step on the frozen formulation; and

effecting a secondary drying step on the frozen formulaiton.

In this procedure, the storage stabilizer may be any of the materials discussed above.

The freeze drying steps of the procedure may be affected in the following manner. The freezing step is effected on said formulation to a temperature of about −30° C. to about −60° C. and said primary and secondary drying steps are effected while raising the temperature of the frozen formulation first to a temperature of about −15° C. to about 45° C. and holding at that temperature and then to a temperature of about 15° C. to about 30° C. and holding at that temperature. The freeze drying steps may be effected under specific sets of conditions as set forth in Table 5 below, particularly effecting the steps under the conditions of Cycle 14 in Table 5 below effected on the formulation F8 of Table 4.

The hosts protected against disease caused by RSV include humans and the invention includes methods of immunization and protection of hosts against disease caused by infection by RSV by administering the immunogenic and preparations and vaccines as provided herein to susceptible hosts. The hosts may be elderly humans or other humans previously exposed to RSV and immunologically primed to respond to the immunization.

### BRIEF DESCRIPTION OF DRAWINGS

FIG. 1 shows a flow diagram of a process used to purify RSV subunits from virus infected cells.

FIG. 2, consisting of panels A, B, and C, illustrates protein stability of an embodiment of the present invention (preparation F8 containing stabilizer) as measured by ELISA over 8 weeks at 25° C. (o) and 37° C. (□) for RSV F, panel A, RSV G, panel B and RSV M, panel C, compared to a sample of the same RSV immunogenic preparation in the absence of stabilizer (“unformulated”).

FIG. 3, consisting of panels A and B, illustrates in panel A, an SDS-PAGE gel of the RSV formulation of FIG. 2 after 3 weeks of incubation at 25° and 37° C., and the corresponding western blot in panel B probed with mouse monoclonal antibodies to RSV F, RSV G and a rabbit mono-specific polyclonal antibody to RSV M.

FIG. 4, consisting of panels A and B, illustrates in panel A, an SDS-PAGE gel of the RSV formulation of FIG. 2 after 8 weeks of incubation at 25° and 37° C., and the corresponding western blot in panel B probed with the same antibodies as described in FIG. 3.

FIG. 5, consisting of panels A and B, illustrates in panel A, an SDS-PAGE gel of the RSV formulation of FIG. 2 after 11 months incubation at 2° to 8° C., and the corresponding western blot in panel B probed with the same antibodies as described in FIG. 3.

FIG. 6, consisting of panels A and B, illustrates in panel A, an SDS-PAGE gel of the RSV formulation of FIG. 2 after 17 months incubation at 2° to 8° C., and the corresponding western blot in panel B probed with the same antibodies as described in FIG. 3.

### GENERAL DESCRIPTION OF THE INVENTION

As discussed above, the present invention provides sub-unit vaccines against disease caused by infection by RSV. The vaccines are not adjuvanted, by which is meant they do not contain extrinsic adjuvants, such as alum. In a preferred embodiment, proteins to be included in the sub-unit vaccines include the RSV F, G and M proteins. The proteins can be isolated from a strain of RSV by, for example, immunoaffinity purification, ion-exchange or other biochemical procedures as described in, for example, the aforementioned WO 94/27636 or by the procedure described in U.S. Pat. No. 5,194,595. The proteins contained in the sub-unit vaccines may be present as a co-isolated and co-purified mixture of RSV F, G and M proteins of RSV and may be isolated as described in the aforementioned U.S. Pat. No. 6,020,182. (Each of the cited patent documents are incorporated herein by reference thereto).

The RSV proteins and immunogenic fragments thereof can be isolated from recombinant organisms that express the proteins or immunogenic fragments. The gene encoding the F protein is described in ref. 20. The gene encoding the G protein is described in ref. 21 and the gene encoding the M protein is described in ref. 22. The production of recombinant organisms expressing the RSV proteins or immunogenic fragments thereof and the identification and purification of the expressed gene products is described in, for example, U.S. Pat. No. 5,223,254 (and incorporated herein by reference thereto). Such recombinants include any bacterial transformants, yeast transformants, cultured insect cells infected with recombinant baculoviruses or cultured mammalian cells as known in the art, for example, Chinese hamster ovary cells that can express the RSV virus proteins or immunogenic fragments thereof.

The RSV proteins and immunogenic fragments thereof can also be chemically synthesized.

The fusion (F) protein may comprise multimeric fusion (F) proteins which may include, when analyzed under nonreducing conditions, heterodimers of molecular weight approximately 70 kDa and dimeric and trimeric forms thereof.

The attachment (G) protein may comprise, when analyzed under non-reducing conditions, oligomeric G protein, G protein of molecular weight approximately 95 kDa and G protein of molecular weight approximately 55 kDa.

The matrix (M) protein may comprise, when analyzed under non-reducing conditions, protein of molecular weight approximately 28 to 34 kDa.

The immunogenic compositions provided herein may be formulated as a vaccine for in vivo administration to a host, which may be a primate, most preferably a human host, to confer protection against disease caused by RSV. The immunogenic compositions and vaccines provided herein may comprise at least one further immunogenic material, which may be an antigen from a pathogen other than RSV, such as a bacterial or viral antigen, to provide a combination vaccine for protection against a plurality of diseases.

As set forth in more detail in the Examples, vaccines comprising the RSV F, G and M proteins were formulated as vaccines and administered to humans in a clinical trial. Prior to immunization (day 0) and then on day 32, day 60 and day 180 postiminunization, serum samples were obtained from the vaccinees. These samples were assayed for:

anti-F antibodies;

anti-G antibodies; and

neutralizing antibodies (NA) against RSV A and RSV B strains.

The antibody titers obtained following immunization with the vaccines as provided herein are shown in Tables 1 to 3. The vaccines were immunogenic and elicited high anti-F, anti-G antibodies and, in particular, were able to neutralize both RSV A and RSV B viruses. Unexpectedly, surprisingly and contrary to decades of RSV vaccine research and development, it was discovered that there was no requirement for an adjuvant in sub-unit vaccines that can protect against disease caused by RSV infection, as demonstrated by the immunogenicity and the ability of the non-adjuvanted vaccines to elicit increased virus-neutralizing antibodies in humans susceptible to disease caused by RSV infection. In the Tables below, GMT refers to geometric mean titre and N refers to sample size.

Vaccination of expectant mothers (active immunization) can be employed to protect young children by passive transfer of immunity, either transplacentally, or through the mother's milk.

In conducting the studies referred to above, it was found that the proteins, when formulated in the absence of an adjuvant, were susceptable to loss of ELISA stability upon storage, possibly due to changes in protein conformation which may lead to loss of effectiveness of the vaccine upon long-term storage. The lack of stability was most pronounced for the M protein.

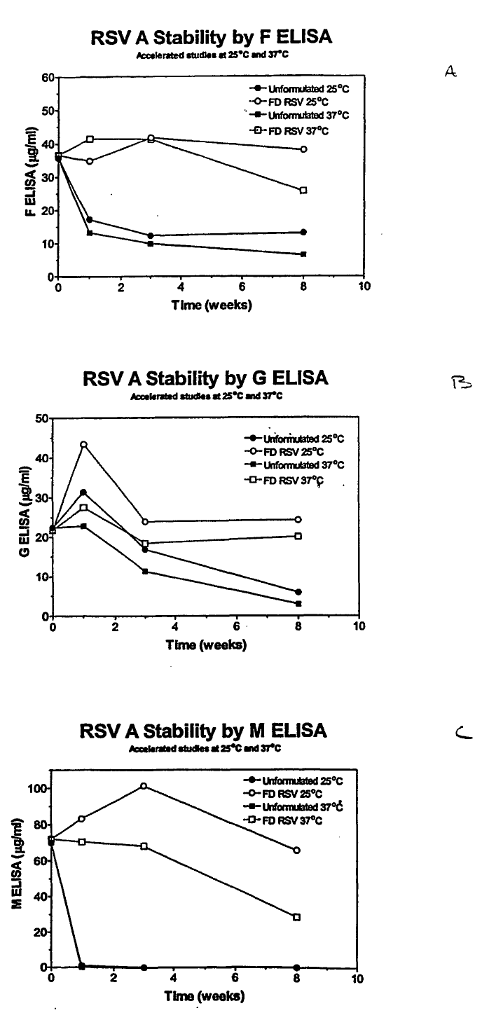

The addition of a storage stabilizer, particularly sucrose, to the composition was demonstrated to prevent the loss of ELISA activity (see FIG. 2) when associated with a freeze-thaw operation to lypholize the composition.

### VACCINE PREPARATION AND USE

Immunogenic compositions, suitable to be used as vaccines, may be prepared from mixtures comprising immunogenic F, G and M proteins of RSV. The immunogenic composition elicits an immune response which produces antibodies, and/or cell mediated responses, such as cytotoxic T-cell response to the specific immunogens.

Immunogenic compositions including vaccines may be prepared as injectables, as liquid solutions, suspensions or emulsions. The active immunogenic ingredients may be mixed with pharmaceutically acceptable excipients which are compatible therewith.

Such excipients may include water, saline, dextrose, glycerol, ethanol and combinations thereof. The immunogenic compositions and vaccines may further contain auxiliary substances, such as, wetting or emulsifying agents, pH buffering agents, to enhance the effectiveness thereof. Immunogenic compositions and vaccines may be administered parentally, by injection subcutaneous, intradermal or intramuscularly injection. Alternatively, the immunogenic compositions formulated according to the present invention, may be formulated and delivered in a manner to evoke an immune response at mucosal surfaces. Thus, the immunogenic composition may be administered to mucosal surfaces by, for example, the nasal or oral (intragastric) routes. Alternatively, other modes of administration including suppositories and oral formulations may be desirable. For suppositories, binders and carriers may include, for example, polyalkalene glycols or triglycerides. Such suppositories may be formed from mixtures containing the active immunogenic ingredient(s) in the range of about 10%, preferably about 1 to 2%. Oral formulations may include normally employed carriers, such as, pharmaceutical grades of saccharine, cellulose and magnesium carbonate. These compositions can take the form of solutions, suspensions, tablets, pills, capsules, sustained release formulations or powders and contain about 1 to 95% of the active ingredients, preferably about 20 to 75%.

The immunogenic preparations and vaccines are administered in a manner compatible with the dosage formulation, and in such amount as will be therapeutically effective, immunogenic and protective. The quantity to be administered depends on the subject to be treated, including, for example, the capacity of the individual's immune system to synthesize antibodies, and, if needed, to produce a cell-mediated immune response. Precise amounts of active ingredients required to be administered depend on the judgment of the practitioner. However, suitable dosage ranges are readily determinable by one skilled in the art and may be of the order of micrograms to milligrams of the active ingredients per vaccination. Suitable regimes for initial administration and booster doses are also variable, but may include an initial administration followed by subsequent booster administrations. The dosage may also depend on the route of administration and will vary according to the size of the host.

The concentration of the active ingredients in an immunogenic composition according to the invention is in general about 1 to 95%. A vaccine which contains antigenic material of only one pathogen is a monovalent vaccine.

### EXAMPLES

The above disclosure generally describes the present invention—A more complete understanding can be obtained by reference to the following specific Examples. These Examples are described solely for purposes of illustration and are not intended to limit the scope of the invention. Changes in form, and substitution of equivalents are contemplated as circumstances may suggest or render expedient. Although specific terms have been employed herein, such terms are intended in a descriptive sense and not for purposes of limitation.

Methods of determining tissue culture infectious dose₅₀ (TCID₅₀/mL), plaque and neutralization titres, not explicitly described in this disclosure are amply reported in the scientific literature and are well within the scope of those skilled in the art. Protein concentrations were determined by the bicinchoninie acid (BCA) method as described in the Pierce Manual (23220, 23225; Pierce Chemical company, U.S.A.), incorporated herein by reference.

CMRL 1969 culture medium was used for cell culture and virus growth. The cells used in this study are vaccine quality African green monkey kidney cells (VERO lot M6) obtained from Institut Merieux. The RS viruses used were the RS virus subtype A (Long and A2 strains) obtained from the American Type culture Collection (ATCC) for use in the virus neutralization assay and a recent subtype A clinical isolate for viral protein purification.

### Example 1

This Example illustrates the production of RSV on a mammalian cell line on microcarrier beads in a 150 L controlled fermenter.

Vaccine quality African Green monkey kidney cells (VERO) at a concentration of 10⁵ cells/mL were added to 60 L of CMRL 1969 medium, pH 7.2 in a 150 L bioreactor containing 360 g of Cytodex-1 microcarrier beads and stirred for 2 hours. An additional 60 L of CMRL 1969 was added to give a total volume of 120 L. Fetal bovine serum was added to achieve a final concentration of 3.5%. Glucose was added to a final concentration of 3 g/L and L-glutamine was added to a final concentration of 0.6 g/L. Dissolved oxygen (40%), pH (7.2), agitation (36 rpm), and temperature (37° C.) were controlled. Cell growth, glucose, lactate and glutamine levels were monitored. At day 4, the culture medium was drained from the fermenter and 100 L of E199 media (no fetal bovine serum) was added and stirred for 10 minutes. The fermentor was drained and filled again with 120 L of E199. The RSV inoculum was added at a multiplicity of infection (M.O.I.) of 0.001 and the culture was then maintained for 3 days before one-third to one-half of the medium was drained and replaced with fresh medium. On day 6 post-infection, the stirring was stopped and the beads allowed to settle. The viral culture fluid was drained and filtered through a 20 μm filter followed by a 3 μm filter prior to further processing.

The clarified viral harvest was concentrated 75- to 150-fold using tangential flow ultrafiltration with 300 NMWL membranes and diafiltered with phosphate buffered saline containing 10% glycerol. The viral concentrate was stored frozen at −70° C. prior to further purification.

### Example 2

This Example illustrates the process of purifying RSV sub-unit from a viral concentrate.

A solution of 50% polyethylene glycol-8000 was added to an aliquot of virus concentrate prepared as described in Example 1to give a final concentration of 6%. After stirring at room temperature for one hour, the mixture was centrifuged at 15,000 RPM for 30 min in a Sorvall SS-34 rotor at 4° C. The viral pellet was suspended in 1 mM sodium phosphate, pH 6.8, 2 M urea, 0.15 M NaCl, stirred for 1 hour at room temperature, and then re-centrifuged at 15,000 RPM for 30 minutes in a Sorvall SS-34 rotor at 4° C. The viral pellet was then suspended in 1 mM sodium phosphate, pH 6.8, 50 mM NaCl, 1% Triton X-100 and stirred for 30 minutes at room temperature. The insoluble virus core was removed by centrifugation at 15,000 RPM for 30 min. in a Sorval SS-34 rotor at 4° C. The soluble protein supernatant was applied to a column of ceramic hydroxyapatite (type II, Bio-Rad Laboratories) and the column was then washed with five column volumes of 1 mM sodium phosphate, pH 6.8, 50 mM NaCl, 0.02% Triton X-100. The RSV sub-unit composition, containing the F, G and M proteins, was obtained by eluting the column with 10 column volumes of 1 mM sodium phosphate, pH 6.8, 400 mM NaCl, 0.02% Triton X-100.

### Example 3

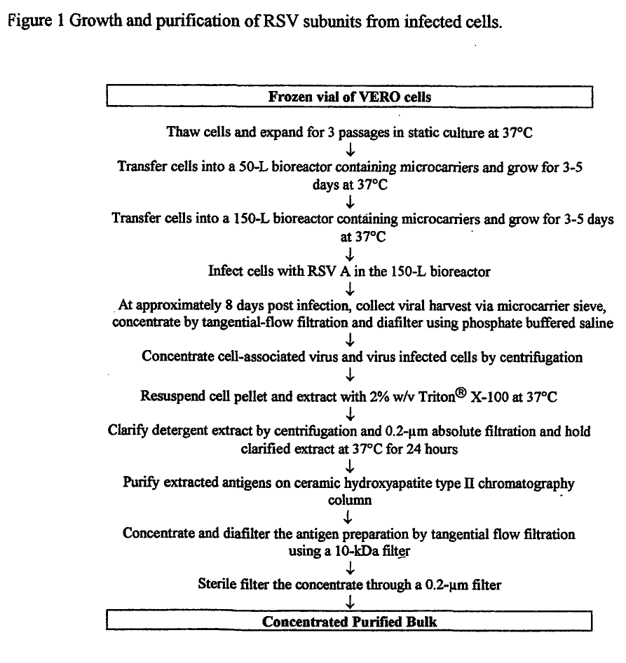

This Example illustrates growing and purifying RSV sub-units from infected cells (see FIG. 1).

VERO cells (Lot LS-7) were grown for 3 passages in static culture in medium (CMRL 1969) containing 10% v/v FBS. The cells were then transferred to a 50-L bioreactor containing microcarriers and to T150 control cell flasks in medium (CMRL 1969) containing 3.5% v/v FBS and incubate for 3 to 5 days at 37° C. These cells were then transferred to a 150-L bioreactor containing microcarriers in medium containing 3.5% v/v FBS and incubate for 3 to 5 days at 37° C. After 34 days of growth at 37° C. in the 150-L bioreactor, the microcarriers are allowed to settle and the growth medium was removed. The cells were then washed once with serum-free medium and the microcarriers were allowed to settle and the medium removed. The cells were then infected with RSV A in 1500 L serum-free medium. After 3 to 4 days post-infection, the microcarriers are allowed to settle, and half of the volume of medium was replaced with serum-free medium. The cells were then incubated for a further 4 to 6 days at 37° C.

The cells were then harvested and filtered through a 100 μm sieve and washed with 500 L of PBS. The microcarrier-free material was collected in a holding tank and concentrated by tangential flow filtration on a 500-kDa filter membrane. This material was concentrated approximately 20-fold and diafiltered using Dulbecco's PBS.

The virus infected cells and cell associated virus were then collected by batch centrifugation for 30 minutes at 5,000×g. The pellet was resuspend in 10 mM sodium. phosphate buffer, containing 300 mM NaCl. The resuspended pellet was then extracted with 2% w/v Triton® X-100 and stirred at 35° to 39° C. for 1 hour. The extract containing soluble F, G and M viral proteins was then clarified the extract by centrifugation for 60 min at 25,000×g. The supernatant was then diluted 3- to 5-fold with 2% w/v Triton® X-100 solution and further clarified by filtration through an absolute 0.2-μm filter.

The filtered extract was then maintained at 35-39° C. for 24 hours with mixing for RSV virus inactivation. To the extract, 2% w/v Triton®X-100 was added to dilute the supernatant 10-fold as compared to initial volume of supernatant. The extract containing F, G and M proteins was then loaded onto a ceramic hydroxyapatite type II chromatography column and the column equilibrated with 1 mM sodium phosphate buffer, containing 30 mM NaCl and 0.02% w/v Triton® X-100.

F, G and M proteins were then eluted with 1 mM sodium phosphate buffer, containing 550 mM NaCl and 0.02% w/v Triton® X-100 and concentrated by ultrafiltration on a 10-kDa filter membrane and diafiltered with 10 mM sodium phosphate buffer, containing 150 mM NaCl and 0.01% w/v Triton® X-100. The resulting solution containing F, G and M proteins was sterilized using a 0.2 μm absolute filter. This represents the concentrated purified bulk (FIG. 1).

The concentrated bulk had a composition distribution:

| | |
|---|---|
| F glycoprotein | 48 wt % |
| G glycoprotein | 5 wt % |
| M Protein | 42 wt % |
| Protein impurities | 5 wt % |

### Example 4

This Example describes the formulation of vaccines and testing in humans.

RSV sub-unit preparations, produced according to Example 3, were used to formulate a non-adjuvanted vaccine, an alum-adjuvanted vaccine and a placebo control that contained only alum. The total protein present in a single dose of the vaccines of the antigens RSV F, G, and M was 100 μg, present in 0.5 mL of phosphate buffered saline. In the alum-adjuvanted vaccine, there was 1.5 mg of alum per 0.5 mL of vaccine.

The vaccines were assessed for stability for 42 months at 5° C., 5 months at 25° C. and 5 weeks at 37° C. to ensure physical and biological stability over time. Stability studies indicated that the F and G antigens in the non-adjuvanted vaccines are stable at 25° C. for at least 6 weeks.

The vaccine preparations were used to immunize adults, 65 years of age or older. Blood samples were obtained on day 0 (day of immunization), day 32, day 60 and day 180, RSV serology was performed on the serum samples as follows:

RSV neutralization assays by a plaque reduction method (NA) against RSV A and RSV B as follows:

1. A colourmetric 96-well plaque reduction assay in tissue culture cells was performed on human sera to assess the neutralization titre. The titre is defined as the amount of human sera required to neutralize 60% of a standard RSV A virus sample. The assay is based on Prince et al.,(ref.23).

The sera were heat-inactivated at 56° C. for 30 minutes. The samples were then diluted in 3-fold serial steps in a 96-well plates and mixed with an equal volume of RSV A (Long strain 30 to 70 pfu) in assay media containing 10% guinea pig complement.

After incubation for 1 hour at 37° C., the mixture was inoculated onto VERO cells for 1 to 2 hours. The inoculum was then removed and the VERO cells overlaid with 0.75% methylcellulose and incubated for 4 to 5 days. After the 4-day incubation, the cells were fixed with a mixture of 2% formaldehyde and 0.2% glutaraldehyde. Viral plaques were then visualized by immunostaining using a monoclonal antibody to the RSV F protein, followed by a donkey anti-mouse IgG F(ab′)2 -horseradish peroxidase conjugate. The enzyme substrates were tetramethylbenzidirine (TMB) and hydrogen peroxide. The neutralization titre is expressed as the reciprocal of the dilution which results in 60% reduction in plaque formation as determined by linear interpolation analysis. (Tables 1 to 3).

2. F glycoprotein-specific antibodies by enzyme linked immunoassay (ELISA); Enzyme linked immunosorbert assays (ELISA) are generally known in the art. Briefly, this ELISA assay is for the detection and quantitation of human IgG antibodies to the Fusion (F) protein of Respiratory Syncytial Virus A (RSVA F). The assay utilizes microtitre plates coated with purified RSV-F antigen to sequester F-specific IgG antibodies and peroxidase-coupled antibodies to human IgG as the indicator.

Microtitre plates were coated with purified RSV-F antigen for 16 to 24 hours. The coating solution was blotted, and the plates were incubated with a blocking solution and then washed. Dilutions of serum standard, control sera and test samples were added to the wells. The plates were incubated and washed. Horseradish peroxidase (HRP)-conjugated anti-human IgG was added at the working dilution. The plates were incubated and washed again. Tetramethyl benzidine (TMB) was diluted to the working concentration in hydrogen peroxide (H₂O₂) was added and the plates were incubated further. The reaction was quenched with 1 M sulphuric acid (H₂SO₄) and the colour reaction measured by reading the optical density (O.D.) of each well.

In this procedure, a test sample containing IgG antibodies to RSV-F forms a 3-layer sandwich attached to the solid phase (microtitre plate). The intensity of colour development in each well is directly proportional to the amount of anti-human IgG peroxidase attached to the solid phase and, therefore, to the anti-RSV-F IgG content of the test sample. To quantitate the amount of anti-RSV-F IgG in each test sample, eight (8) 2-fold dilutions of each sample are tested against a serially diluted standard. Two controls, a positive and a negative, are included on each plate. Antibody levels are expressed in ELISA units (E.U.), obtained by assigning 100,000 E.U. to the Serum Standard.

3. G glycoprotein-specific antibodies were measured by enzyme linked immunoassay (ELISA). Briefly this ELISA assay is for the detection and quantitation of human IgG antibodies to the attachment glycoprotein (G) of Respiratory Syncytial Virus (RSV). The assay utilizes microtitre plates coated with purified RSV-G antigen to bind G-specific IgG antibodies and peroxidase-coupled antibodies to human IgG as the indicator.

Microtitre plates were coated with purified RSV-G antigen for 16 to 24 hours. The coating solution was blotted, and the plates were incubated with a blocking solution and then washed. Dilutions of serum standard, control sera and test samples were added to the wells. The plates were incubated and washed. Horseradish peroxidase (HRP) conjugated anti-human IgG was added at the working dilution. The plates were incubated and washed again. Tetramethyl benzidine (TMB) diluted to the working concentration in hydrogen peroxide (H₂O₂) was added and the plates were incubated further. The reaction was quenched with 1 M sulphuric acid (H₂SO₄) and the colour reaction measured by reading the optical density (O.D.) of each well.

In this procedure, a test sample containing IgG antibodies to RSV-G forms a 3 layer sandwich attached to the solid phase (microtitre plate). The intensity of colour development in each well is directly proportional to the amount of anti-human IgG peroxidase attached to the solid phase and, therefore, to the antiRSV-G IgG content of the test sample. To quantitate the amount of anti-RSV-G IgG in each test sample, eight (8) 2-fold dilutions of each sample are tested against a serially-diluted standard. Two controls, a positive and a negative, are included on each. plate. Antibody levels are expressed in ELISA units (E.U.), obtained by assigning 100,000 E.U. to the Serum Standard.

The immunogenicity of the vaccine preparation is shown in Table 1 as the geometric mean titer and the 95% confidence intervals for the non-adjuvanted vaccine, the vaccine adjuvanted with alum and the alum control.

Tables 2 and 3 show the number of vaccinees in which there was a greater or equal to 2-fold increase in antibody titer (Table 2) or 4-fold increase in antibody titer (Table 3) compared to pre-immunization titers.

### Example 5

This Example illustrates the stabilization of the RSV vaccines described in Example 4.

The immunogenic preparations of RSV described in Example 4 were formulated as illustrated below. These formulations included the use of stabilizers and freeze-drying.

A purified bulk of RSV proteins (400 μg/ml), prepared as described in Example 3, was mixed with an equal volume of stabilizer solution to provide the final concentration of stablizer shown in Table 4. Vials (2.2 ml) were filled with 0.5 ml of this mixture and stoppered. Vials were then placed in a metallic tray in a Dura Stop MP freeze-dryer (FTS Kinetics) and subjected to various freeze-drying cycles as outlined in Table 5.

Several stabilizer formulations (Table 4) and freeze-drying cycles were performed (see Table 5). The freeze-drying cycle comprised three steps, including a freezing step, a primary drying step, and a secondary drying step. After the freeze-drying procedure, samples were tested by SDS-PAGE, western blot and ELISA assays after varying storage times (1, 3 and 8 weeks) at 25° C. and 37° C. Unformulated non-lyophilized samples of the RSV preparations were stored at −70° C. as control samples. FIG. 2 shows the results of ELISA assays on the formulated RSV proteins (F, G, and M) from the F8 (5% sucrose—Table 4) sample after 8 weeks at 25° C.

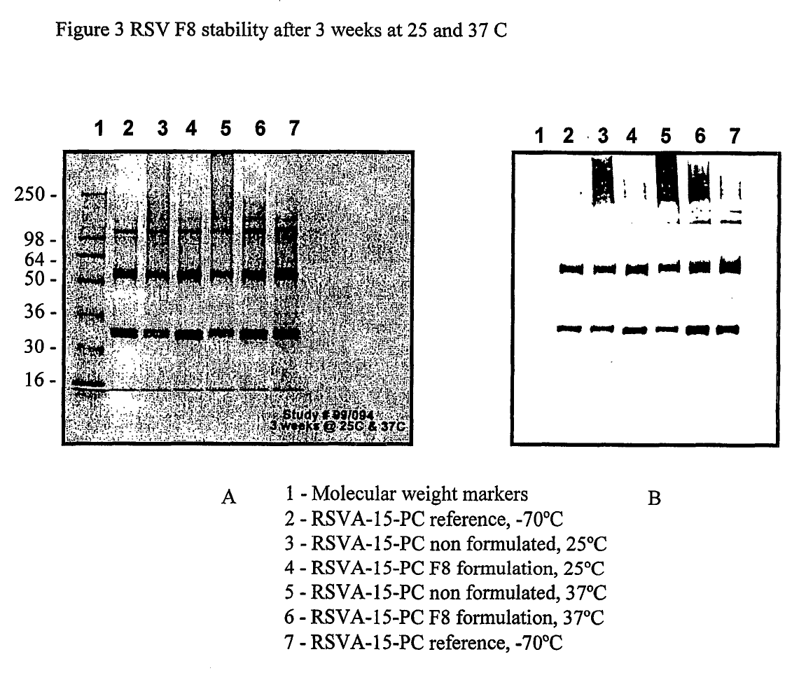

Samples were also analyzed by SDS-PAGE and western blot (FIGS. 3, 4, 5 and 6). These Figures show an SDS-PAGE gel in panel A and the corresponding western blot in panel B. The western blots were probed with mouse monoclonal antibodies against F1 and G proteins, and a rabbit mono-specific polyclonal antibody against M protein.

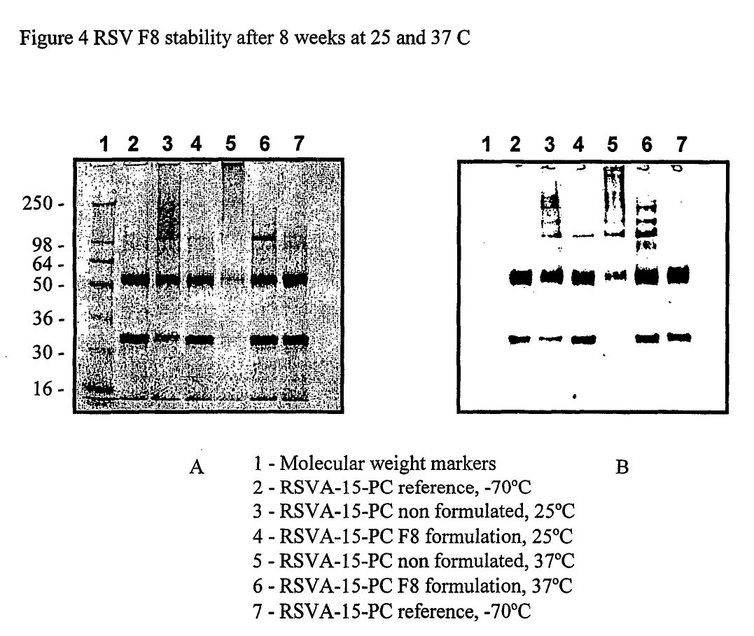

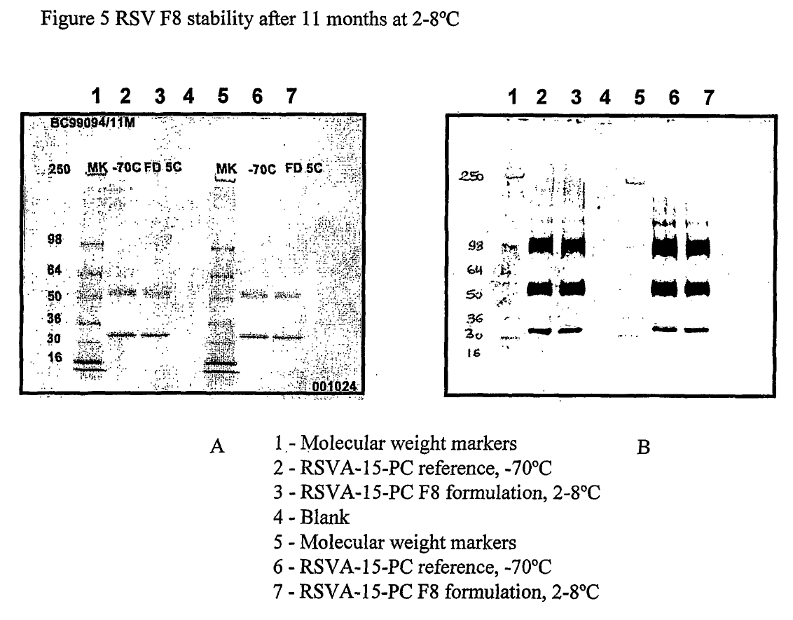

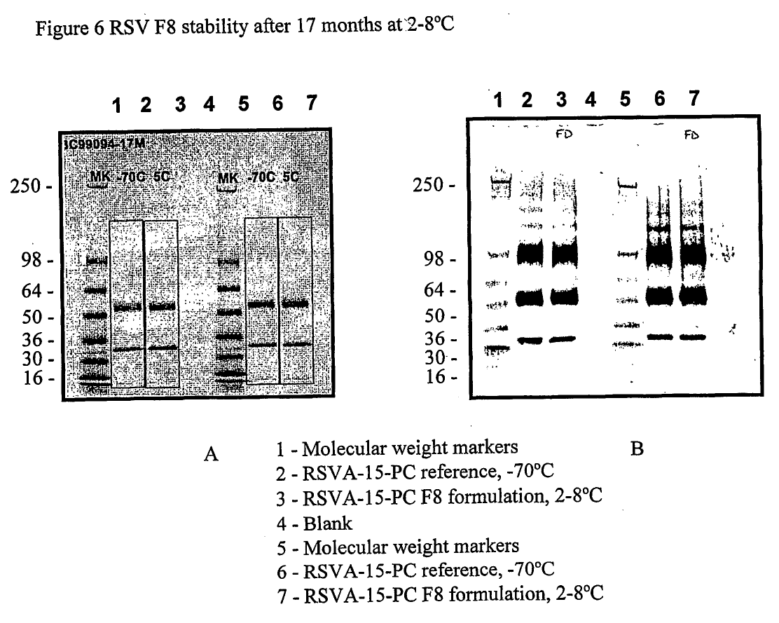

At the elevated temperature of 37 C, similar results were obtained compared to the 25° C. samples after 3 weeks. After 8 weeks (FIG. 4), there was noticeable loss of M protein reactivity in the unformulated sample at 25° C. and substantial loss at 37° C. However, the formulated samples at 25° C. (lane 3) and 37° C. (lane 6) showed little loss of reactivity when compared with the reference control sample (lanes 2 and 7). After 11 months (FIG. 5) and 17 months (FIG. 6) storage at 2° to 8° C., the formulated sample showed very little difference compared to the reference sample. It is difficult to see the band corresponding to the G protein on an SDS-PAGE gel and western blots due to the low content of G in this embodiment and the carbohydrates on the protein. The G band is sometimes visible as a smear above the F band, for example, see FIG. 3 panel B.

### SUMMARY OF THE DISCLOSURE

In summary of this disclosure, the present invention provides non-adjuvanted immunogenic preparations (including vaccines) for protection against disease caused by Respiratory Syncytial Virus (RSV) infection. The immunogenic preparations contain at least one protein of RSV or at least one immunogenic fragment thereof Methods of immunization using the immunogenic preparations are also provided. Various formulations of these preparations are also provided. Modifications are possible within the scope of the invention.

TABLE 1
Serum Antibodies Directed against RSV A and RSV B GMT and 95% Cl

| | | 100 μg dose/no adjuvant | | | 100 μg dose/adjuvant 1.5 mg | | | Control | | |
|---|---|---|---|---|---|---|---|---|---|---|
| Day | Antibody | GMT | Lower | Upper | GMT | Lower | Upper | GMT | Lower | Upper |
| Day 0 | NA to RSV A | 1858.7 | 1588 | 2175.6 | 1987.1 | 1633.5 | 2417.2 | 1818.2 | 1551.5 | 2130.7 |
| Day 0 | NA to RSV B | 1541.9 | 1308 | 1817.6 | 1510.4 | 1246.6 | 1830.1 | 1564.1 | 1348 | 1814.8 |
| Day 0 | Anti-F | 71444.9 | 60512.2 | 84352.8 | 72093.5 | 60307 | 86183.6 | 73234.6 | 62631.9 | 85632.2 |
| Day 0 | Anti-G | 60504.7 | 49172.3 | 74448.8 | 69710.9 | 57795.3 | 84083.1 | 76336.9 | 64091.2 | 90922.5 |
| Day 32 | NA to RSV A | 8699.5 | 7037.4 | 10754.1 | 7627.4 | 6298.9 | 9236 | 1731.4 | 1485.7 | 2017.8 |
| Day 32 | NA to RSV B | 5617.2 | 4629 | 6816.5 | 4994.6 | 4136.9 | 6030.2 | 1552 | 1331.2 | 1809.3 |
| Day 32 | Anti-F | 345753.6 | 284223.8 | 420603.6 | 311418.1 | 262682.4 | 369195.9 | 73542.3 | 62794 | 86130.3 |
| Day 32 | Anti-G | 279002 | 229409 | 339315.8 | 193516.7 | 161887.9 | 231325 | 74111 | 62145.5 | 88380.4 |
| Day 60 | NA to RSV A | 7596.9 | 6212.5 | 9289.9 | 7495.5 | 6277.4 | 8950 | 1808 | 1539.1 | 2123.8 |
| Day 60 | Anti-F | 323125.9 | 265503.6 | 393253.9 | 314135.9 | 267418 | 369015.4 | 75367.1 | 64209 | 88464.2 |
| Day 60 | Anti-G | 259202.1 | 212971.6 | 315468 | 175019 | 147707.2 | 207380.9 | 80217.7 | 67060 | 95957.1 |
| Day 180 | NA to RSV A | 5073.5 | 4245.6 | 6062.8 | 4718.7 | 3936.5 | 5656.3 | 2276 | 1881.9 | 2752.8 |
| Day 180 | Anti-F | 204435.7 | 173651.4 | 240677.5 | 205150.6 | 174134.9 | 241690.6 | 79623.8 | 66378.5 | 95512.1 |
| Day 180 | Anti-G | 155568.7 | 128250.9 | 188705.4 | 126833.4 | 106591.3 | 150919.5 | 74767.5 | 61397.6 | 91048.9 |

TABLE 2
Greater than or Equal to Two Fold increase antibody titre

| | | 100 μg/no | | 100 μg/ | | | |
|---|---|---|---|---|---|---|---|
| | | adjuvant | | adjuvant | | Control | |
| Day | Antibody | N | % | N | % | N | % |
| Day32/Day0 | NA to RSV A | 87 | 80.56 | 86 | 76.11 | 1 | 0.93 |
| Day32/Day0 | NA to RSV B | 78 | 72.22 | 77 | 68.14 | 0 | 0 |
| Day32/Day0 | NA to RSV A | 72 | 66.67 | 70 | 61.95 | 0 | 0 |
| | and RSV B | | | | | | |
| Day32/Day0 | Anti-F | 90 | 83.33 | 92 | 81.42 | 2 | 1.87 |
| Day32/Day0 | Anti-G | 82 | 76.64 | 70 | 61.95 | 5 | 4.67 |
| Day60/Day0 | NA to RSV A | 80 | 74.77 | 88 | 80 | 4 | 3.85 |
| Day60/Day0 | Anti-F | 81 | 75.7 | 97 | 88.18 | 2 | 1.92 |
| Day60/Day0 | Anti-G | 84 | 78.5 | 62 | 56.36 | 5 | 4.81 |
| Day180/Day0 | NA to RSV A | 68 | 65.38 | 63 | 60 | 14 | 14 |
| Day180/Day0 | Anti-F | 68 | 65.38 | 71 | 67.62 | 8 | 8 |
| Day180/Day0 | Anti-G | 63 | 61.17 | 38 | 36.19 | 7 | 7 |

TABLE 3
Greater than or Equal to Four Fold increase in antibody titre

| | | 100 μg/no | | 100 μg/ | | | |
|---|---|---|---|---|---|---|---|
| | | adjuvant | | adjuvant | | Control | |
| Day | Antibody | N | % | N | % | N | % |
| Day32/Day0 | NA to RSV A | 62 | 57.41 | 50 | 44.25 | 0 | 0 |
| Day32/Day0 | NA to RSV B | 48 | 44.44 | 40 | 35.4 | 0 | 0 |
| Day32/Day0 | NA to RSV A | 41 | 37.96 | 35 | 30.97 | 0 | 0 |
| | and RSV B | | | | | | |
| Day32/Day0 | Anti-F | 60 | 55.56 | 52 | 46.02 | 1 | 0.93 |
| Day32/Day0 | Anti-G | 54 | 50.47 | 32 | 28.32 | 0 | 0 |
| Day60/Day0 | NA to RSV A | 50 | 46.73 | 49 | 44.55 | 1 | 0.96 |
| Day60/Day0 | Anti-F | 60 | 56.07 | 52 | 47.27 | 2 | 1.92 |
| Day60/Day0 | Anti-G | 52 | 48.6 | 28 | 25.45 | 0 | 0 |
| Day180/Day0 | NA to RSV A | 27 | 25.96 | 24 | 22.86 | 3 | 3 |
| Day180/Day0 | Anti-F | 28 | 26.92 | 32 | 30.48 | 4 | 4 |
| Day180/Day0 | Anti-G | 27 | 26.21 | 14 | 13.33 | 3 | 3 |

TABLE 4
Stabilizers

| | Lac- | Su- | Sor- | Man- | Dex- | L- | L- | | | Gluta- | L- | L- | | Sodium | | | Freeze- |
|---|---|---|---|---|---|---|---|---|---|---|---|---|---|---|---|---|---|
| Stabi- | tose | crose | bitol | nitol | tran | Arginine- | Cystine- | BME | MEM | mine- | Histi- | Ala- | | Phosphate | Potassium | | drying |
| lizer | (%) | (%) | (%) | (%) | 70 | HCl | HCl | 100x | 100x | Na | dine | nine | Urea | Dibasic | Phosphate | MSG | Cycle |
| 1 | | | 5.00 | 2.5 | 1.5 | 2.14 | 0.08 | 20 | 20 | | | | 0.90 | | | | 1 |
| 1-1 | | | 2.5 | 2.5 | 1.5 | 2.14 | 0.08 | 20 | 20 | | | | 0.90 | | | | 3 |
| 1-2 | | | 5.0 | 2.5 | 1.5 | 2.14 | 0.08 | 20 | 20 | | | | 0.90 | | | | 3 |
| 1-3 | | | 7.5 | 2.5 | 1.5 | 2.14 | 0.08 | 20 | 20 | | | | 0.90 | | | | 3 |
| 1-4 | | | 2.5 | 5.0 | 1.5 | 2.14 | 0.08 | 20 | 20 | | | | 0.90 | | | | 3 |
| 1-5 | | | 5.0 | 5.0 | 1.5 | 2.14 | 0.08 | 20 | 20 | | | | 0.90 | | | | 3 |
| 1-6 | | | 7.5 | 5.0 | 1.5 | 2.14 | 0.08 | 20 | 20 | | | | 0.90 | | | | 3 |
| 1-7 | | | 2.5 | 10 | 1.5 | 2.14 | 0.08 | 20 | 20 | | | | 0.90 | | | | 3 |
| 1-8 | | | 5.0 | 10 | 1.5 | 2.14 | 0.08 | 20 | 20 | | | | 0.90 | | | | 3 |
| 1-9 | | | 7.5 | 10 | 1.5 | 2.14 | 0.08 | 20 | 20 | | | | 0.90 | | | | 3 |
| 1-10 | | | | 10 | 1.5 | 2.14 | 0.08 | 20 | 20 | | | | 0.90 | | | | 7, 9 |
| 1-11 | | | | 5 | 1.5 | 2.14 | 0.08 | 20 | 20 | | | | 0.90 | | | | 7, 9 |
| 2 | | 2.5 | 2.5 | 1.25 | 1.5 | 1.0 | 0.08 | 10 | 10 | 0.10 | 0.25 | | 0.25 | 0.5 | 0.25 | | 1 |
| 2-1 | | | 2.5 | 2.5 | 1.5 | 1.0 | 0.08 | 10 | 10 | 0.10 | 0.25 | | 0.25 | 0.5 | 0.25 | | 3 |
| 2-2 | | | 5.0 | 2.5 | 1.5 | 1.0 | 0.08 | 10 | 10 | 0.10 | 0.25 | | 0.25 | 0.5 | 0.25 | | 3 |
| 2-3 | | | 10 | 2.5 | 1.5 | 1.0 | 0.08 | 10 | 10 | 0.10 | 0.25 | | 0.25 | 0.5 | 0.25 | | 3 |
| 2-4 | | | 2.5 | 5 | 1.5 | 1.0 | 0.08 | 10 | 10 | 0.10 | 0.25 | | 0.25 | 0.5 | 0.25 | | 3 |
| 2-5 | | | 5.0 | 5 | 1.5 | 1.0 | 0.08 | 10 | 10 | 0.10 | 0.25 | | 0.25 | 0.5 | 0.25 | | 3 |
| 2-6 | | | 10 | 5 | 1.5 | 1.0 | 0.08 | 10 | 10 | 0.10 | 0.25 | | 0.25 | 0.5 | 0.25 | | 3 |
| 2-7 | | | 2.5 | 10 | 1.5 | 1.0 | 0.08 | 10 | 10 | 0.10 | 0.25 | | 0.25 | 0.5 | 0.25 | | 3 |
| 2-8 | | | 5.0 | 10 | 1.5 | 1.0 | 0.08 | 10 | 10 | 0.10 | 0.25 | | 0.25 | 0.5 | 0.25 | | 3 |
| 2-9 | | | 10 | 10 | 1.5 | 1.0 | 0.08 | 10 | 10 | 0.10 | 0.25 | | 0.25 | 0.5 | 0.25 | | 3 |
| 2-10 | | | | 10 | 1.5 | 1.0 | 0.08 | 10 | 10 | 0.10 | 0.25 | | 0.25 | 0.5 | 0.25 | | 7, 9 |
| 2-11 | | | | 5 | 1.5 | 1.0 | 0.08 | 10 | 10 | 0.10 | 0.25 | | 0.25 | 0.5 | 0.25 | | 7, 9 |
| 3 | 7.5 | | 5.0 | 2.50 | 1.50 | 2.14 | 0.08 | 20 | 20 | | | | 0.90 | | | | 1 |
| 5-1 | | 2 | | | | | | | | | | | | | | | 9 |
| 5-2 | | 5 | | | | | | | | | | | | | | | 9 |
| 6-1 | | 2.5 | | 2.5 | | | | | | | | | | | | | 9 |
| 6-2 | | 5.0 | | 5.0 | | | | | | | | | | | | | 9 |
| F1 | | | | | 1.5 | 2.0 | | 20 | 20 | | | | | 0.5 | 0.25 | 0.2 | 11 |
| F2 | | 2.5 | | | 1.5 | 2.0 | | 20 | 20 | | | | | 0.5 | 0.25 | 0.2 | 11 |
| F3 | | 2.5 | | 10 | 1.5 | 2.0 | | 20 | 20 | | | | | 0.5 | 0.25 | 0.2 | 11 |
| F4 | | | | 10 | 1.5 | 2.0 | | 20 | 20 | | | | | 0.5 | 0.25 | 0.2 | 11 |
| F5 | | 2.5 | | | 1.5 | | | | | | | | | 0.5 | 0.25 | | 11 |
| F6 | | 2.5 | | 10 | 1.5 | | | | | | | | | 0.5 | 0.25 | | 11 |
| F7 | | | | 10 | 1.5 | | | | | | | | | 0.5 | 0.25 | | 11 |
| F8 | | 5.0 | | | | | | | | | | | | | | | 11, 14 |
| F9 | | 5.0 | | 5.0 | | | | | | | | | | | | | 11 |
| F10 | | 5.0 | | 5.0 | | | | 20 | 20 | | | | | | | 0.2 | 11 |
| F11 | | 5.0 | | 5.0 | | | | | | | | | | | | 0.2 | 11 |
| F12 | | 5.0 | | | | | | | | | | | | | | 0.2 | 11 |

TABLE 5
Freeze-drying cycle

| Cycle # 1 | Cycle # 3 | Cycle # 7 | Cycle # 9 | Cycle # 11 | Cycle # 14 |
|---|---|---|---|---|---|
| Freeze to −50 C | Freeze to −50 C (2.5 C/min) | Freeze to −40 C | Freeze to −40 C | Freeze to −40 C | Freeze to −50 C |
| (2.5 C/min) | for 240 min | (2.5 C/min) | (2.5 C/min) | (2.5 C/min) | (2.5 C/min) |
| for 250 min | | for 60 min | for 60 min | for 60 min | for 60 min |
| −40 C (2.5 C/min) for | −40 C (2.5 C/min) for 1980 min | −30 C (2.5 C/min) for | −35 C (2.5 C/min) for | −30 C (2.5 C/min) for | −22 C (2.5 C/min) |
| 2000 min | | 1500 min | 1200 min | 1380 min | for 660 min |
| 25 C (2.5 C/min) for | −20 C (2.5 C/min) for 360 min | 25 C (0.1 C/min) for | 25 C (0.5 C/min) for | 25 C (0.25 C/min) | 25 C (0.25 C/min) |
| 240 min | | 720 min | 480 min | for 480 min | for 180 min |
| | −5 C (2.5 C/min) for 60 min | | | | |
| | 0 C (2.5 C/min) for 60 min | | | | |
| | 4 C (2.5 C/min) for 120 min | | | | |
| | 8 C (2.5 C/min) for 60 min | | | | |
| | 12 C (2.5 C/min) for 60 min | | | | |
| | 16 C (2.5 C/min) for 60 min | | | | |
| | 20 C (2.5 C/min) for 60 min | | | | |
| | 25 C (2.5 C/min) for 990 min | | | | |

### REFERENCES

1. Robbins, A., and Freeman, P. (1988). Sci. Am. 259:126-133

2. Glezen, WP., Taber, L. H., Frank, A. L. and Kasel, J. A. (1986) Am. J. Dis. Child. 140:143-146.

3. Katz, S. L., New Vaccine Developments establishing priorities. Vol. 1 Washington:National Academic Press (1985) pp.397-409.

4. Wertz, G. W., Sullender, W. M. (1992) Biotech 20:151-176.

5. Fleming, D, M., Cross K. W. (1993) Lancet 342:1507-10.

6. Falsey A. R., Cunningham C. K., Barker W. H. et al. (1995) J. Infec. Dis. 172:389-94.

7. Dowell S. F., Anderson L. J., Gary H. E. Jr. et al. (1996) J. Infec. Dis. 174:456-62.

8. Minaric-Galinovic, Falsey A. R., Walsh E. E. 1996. Eur. J. Clin. Microbial. Infect. Dis. 15:777-781.

9. Falsey, A. R. (1998) Vaccine 16:1775-1778.

10. Osterweil D., Norman D (1980) J. Am. Geriatrics Soc. 38:659-662.

11. Nicholson K. G., Baker D. J., Farquhar A., Hurd D., Kent J., Smith S. H. (1990) Epidemiol. Infect. 105:609-618.

12. Han L. L., Alexander J. P., Anderson L. J. (1999) Vaccine 179:25-30.

13. Collins, P., McIntosh, K., and Chanock, R. M., in “Fields of Virology” ed. Fields, B. M., Knipe, D. M. and Howley P. M., Lippincott-Raven Press New York, (1996) pp. 13131351.

14. Walsh, E. E., Hall, C. B., Briselli, M., Braudiss, M. W., and Schlesinger, J. J. (1987). J. Infec. Dis. 155:1198-1204.

15. Walsh, E. E., Hruska, J. (1983). J. Virol. 47:171-177.

16. Levine, S., Kleiber-France, R. and Paradiso, P. R. (1987) J. Gen- Virol. 69:2521-2524.

17. Anderson, L. J., Hierholzer, J. C., Tsou, C., Hendry, R. M., Fernie, B. F., Stone Y. and Mcintosh, K. (1985) J. Infec. Dis.151:626-633.

18. Johnson, P. R., Olmsted, R. A., Prince G. A., Murphy, B. R., Ailing D. W., Walsh E. E. and Collins P. L. (1987) J. Virol. 61:3163-3166.

19. Cherrie, A. H., Anderson, K., Wertz, G. W. and Openshaw, P. J. M. (1992) J. Virol. 66:2102-2110.

20. Collins, P. L., Huang, Y. T. and Wertz, G. W. (1984). Proc. Nat). Acad. Sci. 81:7683-7687.

21. Wertz, G. W., Collins P. L., Huang, Y., Gruber C., Levine S., and Ball, L. A. (1985) Proc. Natl. Acad. Sci. 82:4075-4079.

22. Satake, M. and Venkatesan S. (1984). J.Virol. 50:92-99.

23, Prince, G. A., Jenson, A. B., Horswood, R. L., Carmargo, E., Chanock, R. M. (1078). Am. J. Pathol. 93:771-791.

24. Coates, H. N., et al. (1966). Am. J. Epidemeol. 83259-313

25. Johnson, RR., et al. (1987). J. Virol. 61:3163-3166.

1. An immunogenic composition, which comprises at least one protein of Respiratory Syncytial Virus (RSV) or an immunogenic fragment thereof and a pharmaceutically-acceptable carrier therefor, wherein the immunogenic composition is formulated in the absence of an extrinsic adjuvant.

2. The immunogenic composition of claim 1 wherein the at least one RSV protein is from one or both subtypes RSV A or RSV B.

3. The immunogenic composition of claim 1 wherein the at least one RSV protein is selected from the group consisting of RSV F protein, RSV G protein, RSV M protein and immunogenic fragments of the RSV F, G or M proteins.

4. The immunogenic composition of claim 3 wherein the at least one RSV protein is present in said composition in an amount of at about 0.1 μg to about 200 μg per dose.

5. The immunogenic composition of claim 4 wherein the at least one protein comprises a mixture of RSV F protein, RSV G protein and RSV M protein.

6. The immunogenic composition of claim 5 wherein the mixture of RSV proteins is a copurified mixture isolated from a Respiratory Syncytial Virus strain.

7. The immunogenic composition of claim 5 wherein the F, G and M proteins are present in the mixture in the relative proportions of:
F from about 40 to about 70 weight %;
G from about 2 to about 20 weight %;
M from about 20 to about 50 weight %:

8. The immunogenic composition of claim 7 wherein, when analyzed by reduced SDS-PAGE analysis, said fusion (F) protein comprises F₁ of molecular weight approximately 48 kDa and F₂ of molecular weight approximately 23 kDa, said attachment (G) protein comprises a G protein of molecular weight approximately 95 kDa and a G protein of molecular weight approximately 55 kDa, and said matrix (M) protein comprises an M protein of approximately 31 kDa.

9. The immunogenic composition of claim 7 wherein, when analyzed by SDS-PAGE under reducing conditions and silver stained, the ratio of F₁ of molecular weight approximately 48 kDa to F₂ of molecular weight approximately 23 kDa is between 1:1 to about 2:1 by scanning densitometry.

10. The immunogenic composition of claim 7 wherein said mixture consists essentially of said RSV F, G and M proteins.

11. The immunogenic composition of claim 7 wherein the mixture of RSV proteins comprises a coisolated and copurified mixture of non-denatured RSV proteins consisting essentially of the fusion (F) protein, attachment (G) protein and matrix (M) protein of RSV, wherein the mixture is free from lectins and is free from monoclonal antibodies.

12. The immunogenic composition of claim 3 wherein, when analyzed under non-reducing conditions, the F protein comprises heterodimers of apparent molecular weight of about 70 kDa and dimeric and trimeric forms of the RSV F protein.

13. The immunogenic composition of claim 3 wherein, when analyzed under non-reducing conditions, the G protein comprises G protein of molecular weight approximately 95 kDa and G protein of molecular weight approximately 55 kDa and oligomeric G protein.

14. The immunogenic composition of claim 3, wherein, when analyzed by SDS-PAGE under non-reducing conditions, the M protein comprises M protein of molecular weight approximately 28 to 34 kDa.

15. The immunogenic composition of claim 1 devoid of monoclonal antibodies.

16. The immunogenic composition of claim 1 devoid of lectins.

17. The immunogenic composition of claim 1 further comprising a stabilizer against storage degradation of said at least one RSV protein.

18. The immunogenic composition of claim 17 formulated as a freeze-dried preparation.

19. The immunogenic composition of claim 7 further comprising a stabilizer against storage degradation of each of said RSV proteins.

20. The immunogenic composition of claim 19 wherein said stabilizer is selected from the group consisting of mannitol, sorbitol, sucrose and an L amino acid.

21. The immunogenic composition of claim 20 wherein the L-amino acid is selected from the group consisting of L-Arginine-HCl, L-Lysine-HCl, L-Methionine, L-Phenylalanine, L-Tryptophan, L-Tyrosine, L-Asparagine, L-Aspartic acid and L-Glycine.

22. The immunogenic composition of claim 20 wherein the stabilizer is sucrose.

23. The immunogenic composition of claim 22 wherein the sucrose is present in an amount of about 2 to about 10% w/v of the composition.

24. The immunogenic composition of claim 23 formulated as a freeze-dried preparation.

25. The immunogenic composition of claim 1 formulated as a vaccine for in vivo administration to a host to confer protection against RSV.

26. The immunogenic composition of claim 7 formulated as a vaccine for in vivo administration to a host to confer protection against RSV.

27. A method of generating an immune response in a host comprising administering to the host an immunogenic composition of claim 1.

28. A method of generating an immune response in a host comprising administering to the host an immunogenic composition of claim 7.

29. The method of claim 28 wherein the host is a human host.

30. A method of formulating an immunogenic composition of claim 1 comprising the steps of:
formulating said immunogenic composition with a stabilizer against storage degradation of the at least one RSV protein to provide a formulation;
effecting a freezing step on said formulation;
effecting a primary drying step on the frozen formulation; and
effecting a secondary drying step on the frozen formulation.

31. The method of claim 30 wherein said stabilizer is selected from a group consisting of mannitol, sorbitol, sucrose and an L amino acid.

32. The method of claim 31 wherein the L amino acid is selected from the group consisting of L-Arginine-HCl, L-Lysine-HCl, L-Methionine, L-Phenylalanine, L-Tryptophan, L-Tyrosine, L-Asparagine, L-Aspartic acid and L-Glycine.

33. The method of claim 32 wherein the stabilizer is sucrose.

34. The method of claim 33 wherein said sucrose is employed in an amount of between about 2 and about 10% w/v of the formulation.

35. The method of claim 30 wherein said freezing step is effected on said formulation to a temperature of about −30° C. to about −60° C. and said primary and secondary drying steps are effected while raising the temperature of the frozen formulation first to a temperature of about −15° C. to about −45° C. and holding at that temperature and then to a temperature of about 15° C. to about 30° C. and holding at that temperature.

36. The method of claim 30 wherein the freezing step, the primary drying step and the secondary drying step are effected under a set of conditions selected from any one of those defined in Table 5.

37. The method of claim 30 wherein said formulation is formulation F8 of Table 4 and the freezing step, the primary drying step and the secondary drying step are effected under the conditions of Cycle #14 in Table 5.

38. The method of claim 30 which is effected on the immunogenic composition of claim 7.

39. The method of claim 37 which is effected on the immunogenic composition of claim 7.
